(19) United States
(12) Patent Application Publication
(10) Pub. No.: US 2025/0114308 A1
(43) Pub. Date: Apr. 10, 2025

(54) LIPOSOMAL POLYMER BEADLET COMPOSITIONS AND METHODS OF USE THEREOF FOR IMPROVED STABILITY, BIOAVAILABILITY & SUSTAINED RELEASE

(71) Applicant: SpecNova LLC, Boca Raton, FL (US)

(72) Inventor: Sebastian Balcombe, Evergreen, CO (US)

(73) Assignee: SpecNova LLC, Boca Raton, FL (US)

(21) Appl. No.: 18/908,686

(22) Filed: Oct. 7, 2024

Related U.S. Application Data
(60) Provisional application No. 63/588,442, filed on Oct. 6, 2023.

(51) Int. Cl. A61K 9/16; A61K 31/375

(52) U.S. Cl. CPC A61K 9/1617; A61K 9/1652; A61K 9/1658; A61K 9/1664; A61K 31/375

(57) ABSTRACT

Disclosed herein is a composition for sustained release of an active that includes a plurality of liposomal beadlets, wherein each of the plurality of liposomal beadlets comprise:
a liposomal core comprising phosphatidylcholine (PC), phosphatidylethanolamine (PE), and phosphatidylserine (PS) wherein the active is dispersed within the liposomal core; a second core surrounding the liposomal core comprising one or more polypeptide and a polysaccharide; and a polymer beadlet shell surrounding the second core. In certain embodiments, the polymer beadlet shell contains gum arabic; microcrystalline cellulose (MCC); hydroxypropyl methylcellulose (HPMC); glycerol; γ-cyclodextrin; ethyl cellulose; and one or more metal halide salt.

### CROSS-REFERENCE TO RELATED APPLICATION(S)

This application claims priority to U.S. Provisional Application No. No. 63/588,442, filed Oct. 6, 2023, and entitled “LIPOSOMAL POLYMER BEADLET COMPOSITIONS AND METHODS OF USE THEREOF FOR IMPROVED STABILITY, BIOAVAILABILITY & SUSTAINED RELEASE,” which is hereby incorporated by reference in their entirety under 35 U.S.C. § 119(e).

### BACKGROUND

Low bioavailability of many dietary supplements presents a challenge to realizing full potential efficacy. Conversely, the extremely high absorption of some dietary supplements presents a problem when the aim of supplementation is to achieve a sustained and stable increase of the supplement in the body. Rapid spikes in supplement blood levels can exceed the capacity of key transporters to deliver supplements to their cellular targets. Therefore, there is a need in the art for improved compositions and methods to achieve sustain/controlled release of dietary supplements.

### BRIEF SUMMARY

Disclosed herein is a composition for sustained release of an active that comprises a plurality of liposomal beadlets, wherein each of the plurality of liposomal beadlets comprise: a liposomal core comprising phosphatidylcholine (PC), phosphatidylethanolamine (PE), and phosphatidylserine (PS) wherein the active is dispersed within the liposomal core; a second core surrounding the liposomal core comprising one or more polypeptide and a polysaccharide; and a polymer beadlet shell surrounding the second core. In certain embodiments, the polymer beadlet shell contains gum arabic; microcrystalline cellulose (MCC); Hydroxypropyl methylcellulose (HPMC); glycerol; γ-Cyclodextrin; ethyl cellulose; and one or more metal halide salt.

In certain embodiments, the amount of PC in the liposomal core is at least about 50 wt %.

In further embodiments, the amount of PE in the liposomal core is from about 5-8 wt %. In yet further embodiments, the amount of PS in the liposomal core is from about 8-10 wt %. In still further embodiments, the liposomal core has a phase transition temperature of at least about 37° C.

According to certain embodiments, the second core polypeptide comprises pea protein hydrolysate and/or brown rice protein. In certain implementations, the second core polypeptide comprises at least about 10 wt % dipeptides and tripeptides. In further embodiments, the second core polysaccharide comprises sodium hyaluronate, gum Arabic, sodium alginate and/or trehalose.

According to certain embodiments, the second core polysaccharide has a positive charge. In certain alternative embodiments, the second core polysaccharide has a negative charge.

According to certain embodiments, the one or more active is selected from CoQ10; Berberine; Quercetin; Hemp/Cannabinoids, Glutathione; L-Carnitine; Ashwagandha; Rhodiola; Bacopa; vitamin C, and a mushroom extract. In certain exemplary implementations, the active is vitamin C.

In certain embodiments, each of the plurality of beadlets have a diameter of from about 500 to 2000 μm.

Further disclosed herein is a method of providing a subject with sustained release of one or more active by administering to the subject a composition comprising: a plurality of beadlets, wherein each of the plurality of beadlets comprises a plurality of liposomal beadlets, wherein each of the plurality of liposomal beadlets includes a liposomal core comprising phosphatidylcholine (PC), phosphatidylethanolamine (PE), and phosphatidylserine (PS) wherein the active is dispersed within the liposomal core; a second core surrounding the liposomal core comprising one or more polypeptide and a polysaccharide; and a polymer beadlet shell surrounding the second core. In certain implementations, the active is released in the body of the subject over a period of from about 10-12 hours, following oral consumption of the composition by the subject.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows a calibration curve of Vitamin C release, according to certain embodiments.

FIG. 2 shows a curve of Vitamin C release from liposomal slow release beadlets, according to certain embodiments.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles” would either completely lack particles, or so nearly completely lack particles that the effect would be the same as if it completely lacked particles. In other words, a composition that is “substantially free of an ingredient or element may still actually contain such item as long as there is no measurable effect thereof.

Admixing or admixed means the formation of a physical combination of two or more elements which may have a uniform or non-uniform composition throughout and includes, but is not limited to, solid mixtures, solutions and suspensions.

Aqueous and aqueous solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

As used herein, “active” or “bioactive” means the compound in the supplement that is responsible for producing the intended health benefit or therapeutic effect. This “active” component is typically the primary substance that contributes to the supplement's efficacy, such as vitamins, minerals, herbs, amino acids, enzymes, or other nutrients. In certain embodiments, one or more active may be dispersed within the instantly disclosed beadlets.

The present disclosure relates to a novel liposomal polymer beadlet formulation and a novel preparation process where certain actives (e.g., dietary supplements and/or probiotic) bioavailability is increased through allowing a slow release of the bioactive over 8-24 hours, greatly improving the bioactive's stability in the body, allowing more of the bioactive to be available for absorption at a targeted location within the body (e.g., the small intestine). The slow/controlled release of certain bioactives can provide significant benefit. For example, where a dietary ingredient absorbs into the blood too fast and overwhelms/oversaturates the ability of the target cells to transport and/or uptake it, significant quantities of the bioactive are excreted rather than reaching and being absorbed into its target tissue.

Disclosed herein is a composition for sustained release of an active. In certain embodiments, the composition comprises a plurality of liposomal beadlets. In certain embodiments, each of the plurality of beadlets comprises a liposomal core, with an active compound dispersed within the first liposomal core. The first liposomal core is surrounded by a second core comprising polypeptides and a polysaccharide and polymer beadlet surrounding the second core.

According to certain embodiments, the first liposomal core includes phosphatidylcholine (PC), phosphatidylethanolamine (PE), phosphatidylserine (PS), one or more phospholipids, and an active compound dispersed within the core. In certain embodiments, PC constitutes at least about 50 wt % of the first liposomal core. PE is present in the range of about 5-8 wt %, and PS is present in the range of about 8-10wt % in the first liposomal core. In certain implementations, the first liposomal core has a phase transition temperature of at least about 37° C., which ensures stability and controlled release of the active compound.

According to certain embodiments, the second core surrounds the first liposomal core and consists of one or more polypeptides and a polysaccharide. In certain embodiments, polypeptides such as pea protein hydrolysate and/or brown rice protein are used in the second core. In exemplary implementations, the polypeptides contain at least about 10 wt % dipeptides and tripeptides, which further enhance the sustained release properties of the liposomal composition.

According to certain embodiments, the polysaccharide in the second core can be sodium hyaluronate, gum Arabic, sodium alginate, and/or trehalose. The second core polysaccharide may have a positive or negative charge, allowing for customization of the liposomal composition.

According to certain embodiments, the polymer beadlet shell comprises: Gum Arabic; Microcrystalline cellulose (MCC), Hydroxypropyl methylcellulose (HPMC); glycerol, γ-Cyclodextrin, ethyl cellulose; and/or one or more metal halide salt.

In certain embodiments, Gum Arabic comprises about 1-25 wt % of the polymer beadlet shell.

In certain embodiments, MCC comprises about 1-5 wt % of the polymer beadlet shell.

In certain embodiments, HPMC comprises about 1-5 wt % of the polymer beadlet shell.

In certain further embodiments, glycerol comprises about 1-3 wt % of the polymer beadlet shell.

In yet further embodiments, γ-cyclodextrin comprises about 1-3 wt % of the polymer beadlet shell.

In still further embodiments, ethyl cellulose comprises about 1-5 wt % of the polymer beadlet shell.

According to certain embodiments, the one or more metal halide salt comprises sodium chloride. In certain further embodiments, the one or more metal halide salt further comprises potassium chloride. In yet further embodiments, one or more metal halide salt further comprises magnesium chloride. In certain implementations of any of the foregoing embodiments, the one or more metal halide salt comprises from about 0.25-1wt % of the polymer beadlet shell.

According to certain embodiments, each of the plurality of beadlets has a diameter of from about 500 to 2000 μm.

The instantly disclosed composition can be used to deliver numerous actives, including, but not limited to: vitamins such as Vitamin C, Vitamin D, Vitamin E, and various B vitamins; minerals like calcium, magnesium, zinc, and iron; amino acids such as L-arginine, L-glutamine, and L-lysine; omega-3 fatty acids like EPA, DHA, and ALA; probiotics like Lactobacillus acidophilus and Bifidobacterium lactis; antioxidants such as CoQ10, resveratrol, and green tea extract; herbal extracts like ginseng extract, Ginkgo biloba extract, and Echinacea extract; herbal ingredients like ginseng, Ginkgo biloba, and Echinacea; enzymes such as bromelain and papain; fiber sources like psyllium husk and inulin; plant sterols and stanols like beta-sitosterol; prebiotics such as inulin and FOS; adaptogens like ashwagandha and Rhodiola rosea; collagen types I, II, and III; mushroom extracts like reishi extract and lion's mane extract; and fruit extracts and vegetable extracts like blueberry extracts and broccoli extracts.

In certain embodiments, actives suitable for sustained release by the instantly disclosed composition may be any active known in the art for which a sustained/slow release is desirable. In certain embodiments, one or more active is selected from CoQ10; Berberine; Quercetin; Hemp/Cannabinoids, Glutathione; L-Carnitine; Ashwagandha; Rhodiola; Bacopa; and/or a mushroom extract. In further embodiments, the active is a vitamin. In certain embodiments, the vitamin is Vitamin C.

In certain implementations, upon ingestion of the compositions disclosed herein, the active is released over a period of from about 10-12 hours.

In certain embodiments, the polymer beadlet shell further comprises a natural colorant. In exemplary implementations, the natural colorant is selected from paprika extract, turmeric, turmeric extract, beta-carotene, and beet extract. In certain implementations, the natural colorant comprises from about 0.25 to 2.0 wt % of the polymer beadlet shell.

In certain embodiments, the instantly disclosed beadlets enhance subject compliance with a dosing regimen, where a single dose of a dietary ingredient can be taken, rather than multiple times a day.

Various aspects and embodiments of the present disclosure are defined by the following numbered clauses:
1. A composition for sustained release of an active comprising:
a plurality of liposomal beadlets, wherein each of the plurality of liposomal beadlets comprise:
   a liposomal core comprising phosphatidylcholine (PC), phosphatidylethanolamine (PE), and phosphatidylserine (PS) wherein the active is dispersed within the liposomal core;
   a second core surrounding the liposomal core comprising one or more polypeptide and a polysaccharide; and
a polymer beadlet shell surrounding the second core.
2. The composition of clause 1, wherein the polymer beadlet shell comprises:
   Gum Arabic;
   Microcrystalline cellulose (MCC);
   Hydroxypropyl methylcellulose (HPMC);
   glycerol;
   γ-Cyclodextrin;
   ethyl cellulose; and
   one or more metal halide salt.
3. The composition of clause 1, wherein the amount of PC in the liposomal core is at least about 50 wt %.
4. The composition of clause 3, wherein the amount of PE in the liposomal core is from about 5-8 wt %.
5. The composition of clause 4, wherein the amount of PS in the liposomal core is from about 8-10wt %.
6. The composition of clause 5, wherein the liposomal core has a phase transition temperature of at least about 37° C.
7. The composition of clause 1, wherein the second core polypeptide comprises pea protein hydrolysate and/or brown rice protein.
8. The composition of clause 7, wherein the second core polypeptide comprises at least about 10wt % dipeptides and tripeptides.
9. The composition of clause 1, wherein the second core polysaccharide comprises sodium hyaluronate, gum Arabic, sodium alginate and/or trehalose.
10. The liposomal composition of clause 9, wherein the second core polysaccharide has a positive charge.
11. The liposomal composition of clause 10, wherein the second core polysaccharide has a negative charge.
12. The composition of clause 2, wherein the Gum Arabic comprises from about 1-25 wt % of the polymer beadlet shell.
13. The composition of clause 12, wherein MCC comprises about 1-5wt % of the polymer beadlet shell.
14. The composition of clause 13, wherein HPMC comprises about 1-5wt % of the polymer beadlet shell.
15. The composition of clause 1, wherein glycerol comprises about 1-3wt % of the polymer beadlet shell.
16. The composition of clause 1, wherein the γ-cyclodextrin comprises about 1-3wt % of the polymer beadlet shell.
17. The composition of clause 1, wherein the ethyl cellulose comprises about 1-5wt % of the polymer beadlet shell.
18. The composition of clause 1, wherein the one or more metal halide salt comprises sodium chloride.
19. The composition of clause 18, one or more metal halide salt further comprises potassium chloride.
20. The composition of clause 19, one or more metal halide salt further comprises magnesium chloride.
21. The composition of any of clauses 18-20, wherein the one or more metal halide salt comprises from about 0.25-1wt % of the polymer beadlet shell.
22. The composition of clause 1, wherein one or more active is selected from CoQ10; Berberine; Quercetin; Hemp/Cannabinoids, Glutathione; L-Carnitine; Ashwagandha; Rhodiola; Bacopa; and a mushroom extract.
23. The composition of clause 22, wherein the active is vitamin C.
24. The composition of clause 22, wherein the active is a mushroom extract.
25. The composition of clause 24, wherein the mushroom extract turkey tail or lion's mane.
26. The composition of clause 23, wherein the active is released over a period of from about 10-12 hours, following oral consumption.
27. The composition of clause 1, where each of the plurality of beadlets has a diameter of from about 500 to 2000 um.
28. The composition any preceding clause, where the polymer beadlet shell further comprises a natural colorant.
29. The composition of clause 28, the natural colorant is selected from paprika extract, turmeric, beta-carotene, and beet extract.
30. The composition of clause 28, wherein the natural colorant comprises from about 0.25 to 2.0 wt % of the polymer beadlet shell.
31. A method of providing a subject with sustained release of one or more active comprising: administering to the subject a composition comprising: a plurality of beadlets, wherein each of the plurality of beadlets comprises a plurality of liposomal beadlets, wherein each of the plurality of liposomal beadlets comprise:
   a liposomal core comprising phosphatidylcholine (PC), phosphatidylethanolamine (PE), and phosphatidylserine (PS) wherein the active is dispersed within the liposomal core;
   a second core surrounding the liposomal core comprising one or more polypeptide and a polysaccharide; and
a polymer beadlet shell surrounding the second core, wherein the active is released in the body of the subject over a period of from about 10-12 hours, following oral consumption of the composition by the subject.
32. The method of clause 31, wherein the composition is the composition of any preceding clause.

### EXPERIMENTAL

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1—Release Study of Vitamin C Blend Beadlet

The dissolution of immunity beadlets and release of vitamin C at physiological pH (6.8) was studied by preparing simulated intestinal fluid (SIF) without enzymes. The SIF consisted of 13.872 g Potassium dihydrogen phosphate and 35.084 g of disodium hydrogen phosphate and water was added to a final volume of 1000 mL. The final pH was adjusted to 6.8.

About 900 mL of dissolution solution was taken into the vessel. Then degassing the dissolution solution through sonication or other means was important because the presence of dissolved gases may affect results. The beadlet is placed within the dissolution solution in the vessel after the dissolution solution reached sufficient temperature and then the dissolution apparatus (Electronic India, model: 1912) is operated at 50 rpm.

Samples were taken at 1, 4, 8, and 12 hours. About 10 mL of sample solution was collected from the vessel at each time interval. Then the sample solutions were analyzed by a High-Performance Liquid Chromatography (“HPLC”) method.

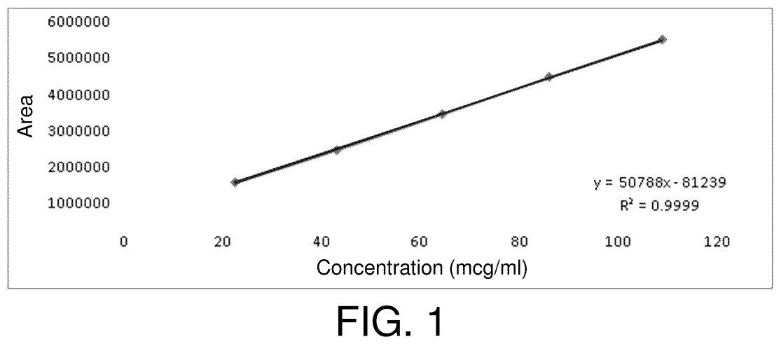

The HPLC used consisted of an LC-10AT VP Solvent Delivery System (Shimadzu), a model SCL-10 A VP System Controller (Shimadzu), and a Model SPD-10AVP UV Detector (Shimadzu). Chromatographic separation was performed on a C18 column at 25° C. The mobile phase was composed of ammonium dihydrogen phosphate (0.05 M, pH 3.0 adjusted by 85 wt wt % phosphoric acid) and acetonitrile at a ratio of 95:5 (v/v), with a flow rate of 1 mL/min. The sample and an analytical standard were injected at a volume of 1 mL each. The detection wavelength for Vitamin C was 245 nm, and the retention time was 5 minutes. To ensure the accuracy of analysis, Vitamin C standard solutions in the range of 10-100 μg/mL were freshly made every time before sample analysis. The concentration of Vitamin C in the samples was determined using the linear regression equation obtained from the respective standard calibration curves of Vitamin C (R2>0.999). An exemplary calibration curve of Vitamin C is shown in FIG. 1

$$wt \% Vitamin C Loading = \frac{Amount of Vitamin C * 100}{Mass of Beadlets} \qquad Eq . 1$$

The HPLC analysis found that average Vitamin C content present in the beadlets was 250 mg with certain overages.

The concentration of Vitamin C in the samples was determined using the respective calibration curves of Vitamin C in SIF (Y=68.512X+1538.2, R2=0.999). To stabilize vitamin C, samples collected at pH 6.8 were also diluted with phosphoric acid (pH 3) before HPLC analysis. The cumulative release percentage of Vitamin C from the beadlets at time t (CR wt %) during in vitro dissolution was determined from the following equations:

$${M}_{s} = \frac{( c * d f )}{{V}_{c}} \qquad Eq . 2$$

$$CR = \frac{c * d f}{V} + {\sum }_{0}^{t - 1} {M}_{s} \qquad Eq . 3$$

$$CR wt \% = ( \frac{CR}{M} ) * 100 \qquad Eq . 4$$

In these equations “c” is the amount of Vitamin C in the withdrawn sample at time t (mg); “D” is the concentration of Vitamin C in the diluted sample analyzed by HPLC at time t (mg/mL); “df” is the dilution factor of sample diluted for HPLC analysis, df=1; “Gc” is the sample volume withdrawn at time t, GC=10 mL; “H” is the cumulative release amount of Vitamin C at time t (mg); G is the volume of dissolution solution, G=900 mL; “Σ₀^{t-1}Mₛ” is the amount of Vitamin C in the withdrawn samples prior to time t (mg); “wt %” is the cumulative release percentage of Vitamin C from a 500 mg beadlet at time t; M is the total amount of Vitamin C in the beadlet (mg).

TABLE 1
Cumulative Release of Vitamin C

| Sr No. | Time (hr) | Area A | Conc. μg/mL | Amt. mg | CDR wt % |
|---|---|---|---|---|---|
| 1 | 1 | 659345 | 14.58 | 72.91 | 29.16 |
| 2 | 4 | 1508690 | 31.31 | 156.53 | 62.61 |
| 3 | 8 | 2256356 | 46.03 | 230.13 | 92.05 |
| 4 | 12 | 2352895 | 47.93 | 239.64 | 95.85 |

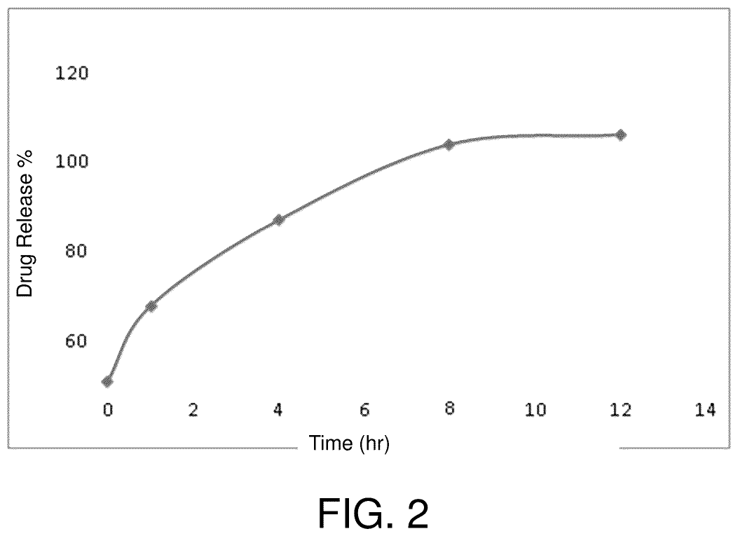

To obtain the rate of Vitamin C release in simulated intestinal fluid (SIF) from beadlets, beadlets with an initial weight of ˜500 mg were incubated in 900 mL of SIF (pH 6.8) solutions with a consistent rotation for a period of 8 hours. It was observed that the beadlets dissolved in SIF within 1 hour. It was observed that a burst release (25wt %) of Vitamin C after 1 hour and could release up to 85wt % up to 8 hours. The release of Vitamin C in SIF showed an initial release of 25wt % in the first hour and 60wt % after 4 hours, and an increase in release up to 90wt % after 8hours. followed by a slight decrease. FIG. 2 shows a release curve.

Although the disclosure has been described with reference to preferred embodiments, persons skilled in the art will recognize that changes may be made in form and detail without departing from the spirit and scope of the disclosed apparatus, systems and methods.

What is claimed is:

1. A composition for sustained release of an active comprising:
a plurality of liposomal beadlets, wherein each of the plurality of liposomal beadlets comprise:
a liposomal core comprising phosphatidylcholine (PC), phosphatidylethanolamine (PE), and phosphatidylserine (PS) wherein the active is dispersed within the liposomal core;
a second core surrounding the liposomal core comprising one or more polypeptide and a polysaccharide; and
a polymer beadlet shell surrounding the second core.

2. The composition of claim 1, wherein the polymer beadlet shell comprises:
Gum Arabic;
Microcrystalline cellulose (MCC);
Hydroxypropyl methylcellulose (HPMC);
glycerol;
γ-Cyclodextrin;
ethyl cellulose; and
one or more metal halide salt.

3. The composition of claim 1, wherein the amount of PC in the liposomal core is at least about 50 wt %.

4. The composition of claim 3, wherein the amount of PE in the liposomal core is from about 5-8 wt %.

5. The composition of claim 4, wherein the amount of PS in the liposomal core is from about 8-10 wt %.

6. The composition of claim 5, wherein the liposomal core has a phase transition temperature of at least about 37° C.

7. The composition of claim 1, wherein the second core polypeptide comprises pea protein hydrolysate and/or brown rice protein.

8. The composition of claim 1, wherein the second core polypeptide comprises at least about 10 wt % dipeptides and tripeptides.

9. The composition of claim 1, wherein the second core polysaccharide comprises sodium hyaluronate, gum Arabic, sodium alginate and/or trehalose.

10. The liposomal composition of claim 9, wherein the second core polysaccharide has a positive charge.

11. The liposomal composition of claim 10, wherein the second core polysaccharide has a negative charge.

12. The composition of claim 2, wherein the Gum Arabic comprises from about 1-25 wt % of the polymer beadlet shell.

13. The composition of claim 1, wherein the ethyl cellulose comprises about 1-5 wt % of the polymer beadlet shell.

14. The composition of claim 1, wherein the one or more metal halide salt comprises sodium chloride, potassium chloride and/or magnesium chloride.

15. The composition of claim 1, wherein one or more active is selected from CoQ10; Berberine; Quercetin; Hemp/Cannabinoids, Glutathione; L-Carnitine; Ashwagandha; Rhodiola; Bacopa; vitamin C, and a mushroom extract.

16. The composition of claim 15, wherein the active is vitamin C.

17. The composition of claim 16, wherein the active is released over a period of from about 10-12 hours, following oral consumption.

18. The composition of claim 1, where each of the plurality of beadlets has a diameter of from about 500 to 2000 μm.

19. A composition for sustained release of an active comprising:
a plurality of liposomal beadlets, wherein each of the plurality of liposomal beadlets comprise:
a liposomal core comprising phosphatidylcholine (PC), phosphatidylethanolamine (PE), and phosphatidylserine (PS) wherein the active is dispersed within the liposomal core, wherein the amount of PE in the liposomal core is from about 5-8 wt % and wherein the amount of PS in the liposomal core is from about 8-10 wt %;
a second core surrounding the liposomal core comprising one or more polypeptide and a polysaccharide; and
a polymer beadlet shell surrounding the second core, wherein the polymer beadlet shell comprises:
Gum Arabic;
microcrystalline cellulose (MCC);
hydroxypropyl methylcellulose (HPMC);
glycerol;
γ-cyclodextrin;
ethyl cellulose; and
one or more metal halide salt.

20. A method of providing a subject with sustained release of one or more active comprising: administering to the subject a composition comprising: a plurality of beadlets, wherein each of the plurality of beadlets comprises a plurality of liposomal beadlets, wherein each of the plurality of liposomal beadlets comprise:
a liposomal core comprising phosphatidylcholine (PC), phosphatidylethanolamine (PE), and phosphatidylserine (PS) wherein the active is dispersed within the liposomal core;
a second core surrounding the liposomal core comprising one or more polypeptide and a polysaccharide; and
a polymer beadlet shell surrounding the second core, wherein the active is released in the body of the subject over a period of from about 10-12 hours, following oral consumption of the composition by the subject.
